(19) United States
(12) Patent Application Publication
(10) Pub. No.: US 2006/0252043 A1
(43) Pub. Date: Nov. 9, 2006

(54) Method for in vitro detection aberrant dysplasia, and the artificial nucleotides being used

(76) Inventors: Hua Bai, Beijing (CN); Yu Sun, Beijing (CN); Jing Zhou, Beijing (CN); Jiyou Li, Beijing (CN); Dajun Deng, Beijing (CN)

Correspondence Address: FOLEY HOAG, LLP;PATENT GROUP, WORLD TRADE CENTER WEST, 155 SEAPORT BLVD, BOSTON, MA, 02110, US

(21) Appl. No.: 10/549,252

(22) PCT Filed: Mar. 13, 2003
(86) PCT No.: PCT/CN03/00180

§ 371 Date: Dec. 5, 2005

(51) Int. Cl. C12Q 1/68; C07H 21/04

(52) U.S. Cl. 435/6; 536/24.3

(57) ABSTRACT

The present invention provides a method for in vitro detection of malignant potential of dysplasia by aberrant methylation of p16 CpG islands, comprising extraction of genomic DNA from a tissue or body fluid sample, analysis of methylation status of p16 CpG islands in the extracted DNA, and evaluation of malignant potential of the tested tissue. The present invention also provides DNA compositions having artificial sequences of p16 CpG islands with or without methylation.

### TECHNICAL FIELD OF THE INVENTION

The present invention relates to detection of malignant potential of dysplasia. The present invention provides a method to measure the potential of malignant progression or transformation of dysplasia, a precancerous lesion of carcinomas, by detection of methylation of p16 CpG islands in DNA of dysplasia lesions. The present invention also provides the artificial DNA sequences corresponding to p16 CpG islands that are used as biomarkers for patients with dysplasia.

### BACKGROUND OF THE INVENTION

Cancer is one of the major causes of human death. Detection, diagnosis, and treatment at early stages of carcinogenesis are very efficient strategies against cancer. Epithelial dysplasia is a precancerous lesion that is observed frequently in various organs such as oral cavity, esophagus, stomach, intestine, liver, etc. It is well known that some dysplasia lesions will progress malignantly. Therefore, the traditional word “dysplasia” is also called “non-invasive neoplasia” in the Padova international classification. However, dysplasia lesions are not always progressive. In fact, they are persistent or regressive in most cases.

Current diagnosis for dysplasia is primarily based on morphologic criteria. Dysplasia lesions are classified as low-grade dysplasia and high-grade dysplasia according to those criteria. The higher the grade, the higher the risk. In a 5-year fellow-up screening, about 3% of low-grade dysplasia transformed malignantly as compared to about 7% of high-grade lesions (You W C, Li J Y, Blot W J, Chang Y S, Jin M L, Gail M H, Zhang L, Liu W D, Ma J L, Hu Y R, Mark S D, Correa P, Fraumeni J F, Xu G W. Evolution of precancerous lesions in a rural Chinese population at high risk of gastric cancer. Int J Cancer 1999; 83: 615-619). It is virtually impossible to identify malignant potential of low-grade dysplasia lesions on histopathologic grounds alone.

The risk of stomach cancer for patients with high-grade gastric dysplasia is up to 100-fold of that for patients with normal gastric mucosa. This results in two contrasting consequences. In one hand, patients with gastric dysplasia are often treated with measures used to treat patients with cancer. For example, stomach resection is administered in some cases. This leads to over-treatment among 90% of patients with dysplasia that does not progress. Therefore, a huge amount of medical resources are wasted and patients are harmed unnecessarily. In another hand, some patients lose their best chance to get the appropriate medical treatment in time, because of their poor social-economic status or ignorance of the high risk of cancer. Thus, means for predicting the malignant potential of dysplastic lesions are eagerly awaited. Such a predictive method will not only save many patients' life, but also bring important economic benefit.

Understanding of carcinogenesis has been advanced significantly and comprehensively by recent developments in the field of molecular biology. It has been observed that loss of function of tumor suppressor genes could result from structural changes of DNA sequences, as well as from changes not related to DNA sequences. The potential structural changes of a target gene are numerous, including point mutation, deletion, insertion, translocation, and amplification. Not all structural changes result in alteration of gene function. The non-structural changes are mainly epigenetic changes, including alteration of methylation patterns of CpG islands, modifications of histones, and chromatin remodeling. Hypermethylation of CpG islands completely silences gene transcription. Hence, it is simpler and more meaningful to detect CpG methylation than to detect structural changes of a target gene for clinical practice. Epigenetic changes play an important role in carcinogenesis.

Methylation-silencing of tumor suppressor gene(s) might result in the formation of malignant potential of a few cells in precancerous lesions such as dysplasia. Detection of the cells with the CpG methylation-silenced gene(s) might be very valuable for prediction of malignant potential of dysplasia. Reverse transcriptional-PCR assay is useful to detect mRNA expression of genes. Immunostaining and Western blot are regular assays for protein products of gene expression. However, results of these assays show the gene expression status existing in the majority of a cell population. Thus, they are not suitable for detecting an abnormal silence of gene expression existing only in a few cells among a cell population in which the majority express the target gene normally.

Some methylation-silenced genes have been used successfully as biomarkers for prediction of malignant transformation. For example, malignant progression of myelodysplastic syndromes was associated with methylation of p15 (Uchida T, Kinoshita T, Nagai H, Nakahara Y, Saito H, Hotta T, Murate T. Hypermethylation of the p15INK4B gene in myelodysplastic syndromes. Blood 1997; 90: 1403-1409; Quesnel B, Guillerm G, Vereecque R, Wattel E, Preudhomme C, Bauters F, Vanrumbeke M, Fenaux P. Methylation of the p 15(INK4b) gene in myelodysplastic syndromes is frequent and acquired during disease progression. Blood 1998; 91: 2985-2990). The majority of microsatellite unstable sporadic colon, endometrium, and stomach cancers have been correlated with transcription silence of hMLH1 by CpG methylation (Hawkins N, Norrie M, Cheong K, Mokany E, Ku S L, Meagher A, O'Connor T, Ward R. CpG island methylation in sporadic colorectal cancers and its relationship to microsatellite instability. Gastroenterology 2002; 122:1376-1387; Esteller M, Catasus L, Matias-Guiu X, Mutter G L, Prat J, Baylin S B, Herman J G. hMLH1 promoter hypermethylation is an early event in human endometrial tumorigenesis. Am J Pathol 1999; 155:1767-1772; Guo R J, Arai H, Kitayama Y, Igarashi H, Hemmi H, Arai T, Hanai H, Sugimura H. Microsatellite instability of papillary subtype of human gastric adenocarcinoma and hMLH1 promoter hypermethylation in the surrounding mucosa. Pathol Int 2001; 51:240-247). Detection of hypermethylation of p15 and hMLH1 are now available in clinical assays. Methylation of p16 was detectable in serum DNA samples from patients with cancer (Esteller M, Sanchez-Cespedes M, Rosell R, Sidransky D, Baylin S B, Herman J G. Detection of aberrant promoter hypermethylation of tumor suppressor genes in serum DNA from non-small cell lung cancer patients. Cancer Res 1999; 59:67-70 Erratum in: Cancer Res 1999; 59:3853; Wong I H, Lo Y M, Zhang J, Liew C T, Ng M H, Wong N, Lai P B, Lau W Y, Hjelm N M, Johnson P J. Detection of aberrant p16 methylation in the plasma and serum of liver cancer patients. Cancer Res 1999; 59:71-73; Sanchez-Cespedes M, Esteller M, Wu L, Nawroz-Danish H, Yoo G H, Koch W M, Jen J, Herman J G, Sidransky D. Gene promoter hypermethylation in tumors and serum of head and neck cancer patients. Cancer Res 2000; 60:892-895). Despite this progress, there has heretofore been no report that the malignant potential of precancerous lesions such as dysplasia could be predicted by detection of the methylation status of p16 CpG islands.

### SUMMARY

The present invention provides a method for in vitro detection of the malignant potential of dysplasia (e.g., of a dysplastic lesion) based on identifying aberrant methylation of p16 CpG islands. The present method comprises extraction of genomic DNA from a tissue or body fluid sample, analysis of methylation status of p16 CpG islands in the extracted DNA, and evaluation of malignant potential of dysplasia in the sampled tissue. The present invention also provides artificial nucleic acids having sequences of p16 CpG islands with or without methylation.

More specifically, the present invention encompasses a method for in vitro detection of malignant potential of dysplasia, comprising the steps of: (a) extraction of genomic DNA from cells from a tissue or body liquid sample; (b) detection of methylation state of p16 CpG islands in the extracted DNA, by amplification with artificial DNAs having the sequences of SEQ ID NOs: 1-4; and (c) evaluation of malignant potential of the tested tissue based upon presence of amplification products corresponding to methylated and unmethylated p16 CpG islands after chemical modification. In some embodiments, the present method is carried out by analyzing the methylation state of p16 CpG islands by methylation-specific PCR (MSP). In speciifc embodiments, the method is carried out using methylated-sequence specific primers that are complementary to any part of the artificial sequence SEQ ID NO: 1 or SEQ ID NO: 3, or using unmethylated-sequence specific primers are complementary to any part of the artificial sequence SEQ ID NO: 2 or SEQ ID NO: 4.

The invention also encompasses the following specific artificial nucleic acids: a first nucleic acid having a sequence corresponding to the antisense sequence of methylated p16 CpG islands depicted in SEQ ID NO: 1, a second nucleic acid having a sequence corresponding to the antisense sequence of unmethylated p16 CpG islands depicted in SEQ ID NO: 2, a third nucleic acid having a sequence corresponding to the sense sequence of methylated p16 CpG islands depicted in SEQ ID NO: 3; and a fourth nucleic acid having a sequence corresponding to the sense sequence of unmethylated p16 CpG islands depicted in SEQ ID NO: 4. The invention also encompasses methylated-sequence specific primer DNA compositions having sequences that are complementary to any part of any of SEQ ID NOs: 1-4.

### BRIEF DESCRIPTION OF THE DRAWINGS

The foregoing and other objects, features, and advantages of the present invention, as well as the invention itself, will be more fully understood from the following description of illustrative embodiments, when read together with the accompanying drawings, in which:

FIG. 1 depicts the result of MSP products of methylated p16 CpG islands from the chemically modified genomic DNA samples extracted from paraffin-embedded tissue blocks.

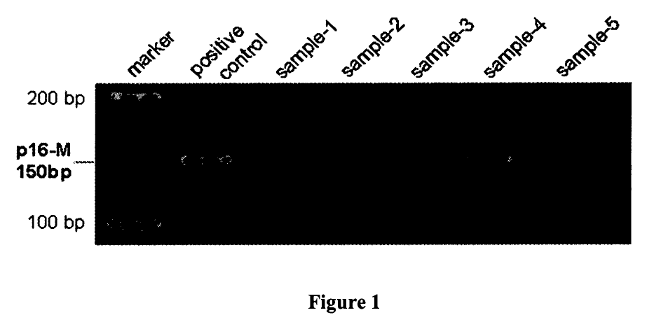

FIG. 2 depicts “SEQ ID NO: 1,” the artificial sequence of the antisense strand of a methylated p16 CpG island with chemical modification. All uracil (U) in the sequence can be replaced by thymidine (t) in PCR products if dTTP is used.

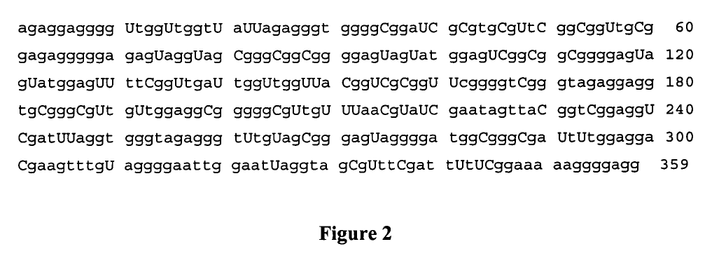

FIG. 3 depicts “SEQ ID NO: 2,” the artificial sequence of the antisense strand of an unmethylated p16 CpG island with chemical modification. All uracil (U) in the sequence can be replaced by thymidine (t) in PCR products if dTTP is used.

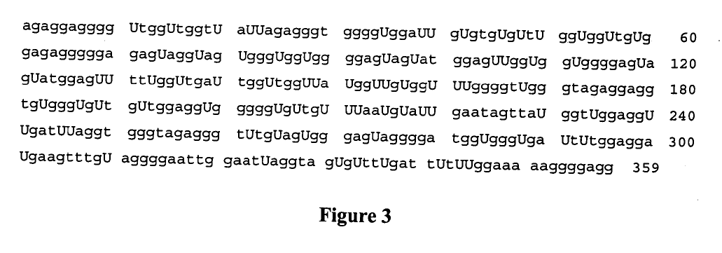

FIG. 4 depicts “SEQ ID NO: 3,” the artificial sequence of the sense strand of a methylated p16 CpG island with chemical modification. All uracil (U) in the sequence can be replaced by thymidine (t) in PCR products if dTTP is used.

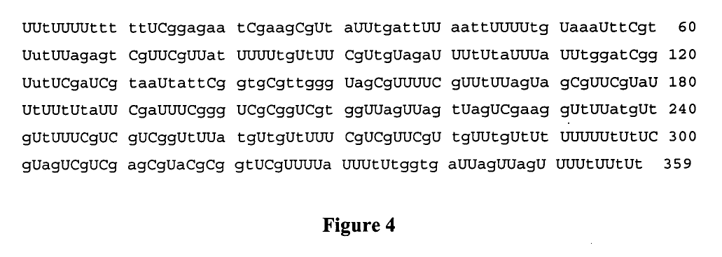

FIG. 5 depicts “SEQ ID NO: 4,” the artificial sequence of the sense strand of an unmethylated p16 CpG island with chemical modification. All uracil (U) in the sequence can be replaced by thyrnidine (t) in PCR products if dTTP is used.

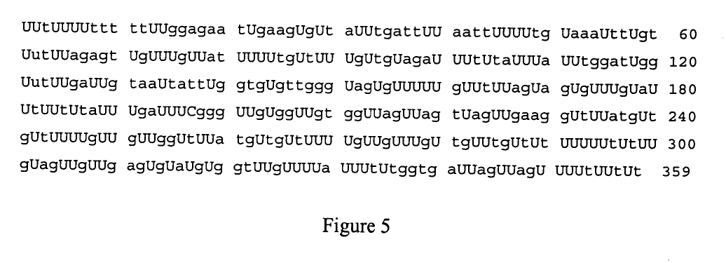

### DETAILED DESCRIPTION

The present invention provides for an in vitro detection assay for malignant potential of dysplasia. The present invention is developed technically according to the following hypothesis and confirmation investigation.

HYPOTHESIS: p16 is an important tumor suppressor gene including a CpG island around its transcriptional start site. Transcription of p16 is regulated by the methylation pattern of the CpG island. The p16 CpG island is unmethylated, enabling regular expression of p16, in normal cells. Aberrant methylation of the CpG island silences p16 transcription. Such abnormal p16 methylation was frequently observed in many kinds of tumors. It has been reported that p16 methylation occurs in 40% of gastric carcinomas (Kang G H, Shim Y H, Jung H Y, Kim W H, Ro J Y, Rhyu M G. CpG island methylation in premalignant stages of gastric carcinoma. Cancer Res 2001; 61: 2847-2851). Our dynamic study on a rat model for gastric carcinogenesis showed that p16 methylation frequency correlated positively with the severity of gastric pathologic lesions. For instance, p16 methylation was found in 2.7% of normal gastric epithelium (n=36), 16.7% of chronic atrophy gastritis (n=24), 37.5% of dysplasia (n=24), 67.4% of gastric adenoma (n=43), and 85.2% of gastric carcinoma (n=27). (Bai H, Gu L K, Zhou J, Deng D J. p16 hypermethylation during gastric carcinogenesis of Wistar rats by N-methyl-N′-nitro-N-nitrosoguanidine. Mutat Res 2003; 535: 73-78). These results suggested that p16 methylation might be an early event whose accumulation ultimately leads to gastric carcinogenesis. Hence, we hypothesized that p16 methylation should be a valuable marker for detection of malignant potential of precancerous dysplastic lesions.

CONFIRMATION: To validate the above hypothesis, we carried out a nested case-control study on a 5-year follow-up endoscopic screen of a high-risk patient population. Aberrant p16 methylation was observed in 5 of 21 samples of dysplasia that progressed to gastric carcinoma, but in none of 21 samples that did not progress (p=0.048, 2-sides). This result proves that presence of the methylated p16 CpG island correlates positively and significantly with malignant progression of gastric dysplasia. In other words, we have found that detection of p16 methylation is a precise assay for predicting the malignant potential of dysplasia. The presence of p16-methylated cells in precancerous lesions such as dysplastic lesions is a strong diagnostic and prognostic indicator of malignant transformation of the lesions.

The present invention provides an assay for the detection of malignant potential of dysplasia comprising the following steps: A) Extraction of genomic DNA from a sample of a target tissue or body fluid from a patient; B) Detection of methylation status of the p16 CpG island in said genomic DNA; and C) Evaluation of malignant potential of the tested tissue.

The present invention for in vitro prediction of malignant potential of dysplasia based on methylation status of p16 CpG islands can be carried out with methylation-specific PCR (MSP, refers to the art-recognized methylation assay described by Herman J G, Graff J R, Myohanen S, Nelkin B D, Baylin S b. Methylation-specific PCR: a novel PCR assay for methylation status of CpG islands. Proc. Natl. Acad. Sci. USA 1996; 93: 9821-9826, and by U.S. Pat. No. 5,786,146). A set of positive control and negative control samples may also be used as quality controls.

The present invention also encompasses a pair of methylated sequence specific MSP primers, which are designed and synthesized according to SEQ ID NO: 1 and SEQ ID NO: 3. These are complementary to chemically modified sequences of the methylated p16 CpG island. The invention further encompasses a pair of unmethylated sequence specific MSP primers, which are designed and synthesized according to SEQ ID NO: 2 and SEQ ID NO: 4. These are complementary to chemically modified sequences of the unmethylated p16 CpG island. The mentioned chemical modification of DNA means deamination of unmethylated cytosines by chemicals such as sodium bisulfite, which converts only unmethylated cytosine to uracil (U) and does not convert methylated cytosines.

The present invention further provides a set of artificial DNA sequences, SEQ ID NO: 1, SEQ ID NO: 2, SEQ ID NO: 3, and SEQ ID NO: 4, which are used to design MSP primers for detection of p16 methylation.

The present invention also provides the following diagnostic criteria for evaluation of malignant potential of dysplastic lesions based on the results obtained by the present assay.

A). When MSP products of methylated p16 CpG island can not be amplified from the chemical modified unmethylated p16 template (negative control) by the methylated-sequence-specific primers, AND when MSP products of methylated p16 CpG island CAN be amplified from the chemically modified templates from the tested lesion at the same time under the same conditions, this lesion is highly malignant.

B). When MSP products of methylated p16 CpG island can be amplified from the chemical modified methylated p16 template (positive control) by the methylated-sequence-specific primers, AND when MSP products of methylated p16 CpG island CANNOT be amplified from the chemical modified templates from the tested lesion, AND when MSP products of unmethylated p16 CpG island can be amplified from the same testing templates, but can not be amplified form the positive control templates, this lesion can not be distinguished from a malignant one.

C). When both MSP products of methylated and unmethylated p16 CpG island CANNOT be amplified from the chemical modified templates from testing lesion by the methylated- and unmethylated-sequence-specific primers, respectively, repeat assay should be carried out till MSP products of methylated OR unmethylated p16 CpG island can be amplified from the testing templates.

The advantages of the present invention include: A) Firstly, it is now proven that the methylated p16 CpG island can be used as biomarker for precise prediction of malignant potential of dysplasia. A very limited number of abnormal cells with methylation-silenced p16 in dysplasia lesion can be detected sensitively by MSP assay. B) Secondly, the invention provides a high specificity and very early prediction. The malignant potential of dysplasia can be detected as early as 5 years before a dysplasia lesion progresses to carcinoma.

Detection of hypermethylation of hMLH1 CpG island is used clinically for the detection of malignant potential of colon polyps as a means of preventing colon cancer. The methylation-silenced p16 is believed to be the second discovered epigenetic biomarker for prediction of malignant potential of precancerous lesions. The prevalence of methylated p16 CpG islands is 20%˜40% of various cancers, which is much higher than that of methylation-silenced hMLH1 (5%˜10%). Therefore, it is reasonable to expect a much wider clincal application of the present invention.

Practice of the invention will be still more fully understood from the following Examples, which are presented solely to illustrate principles and operation of the invention, and should not be limiting scope of the invention in any way.

### EXAMPLES

In Vitro Prediction of Malignant Potential of Gastric Dysplasia by Detection of Modified Sequence of Methylated p16 CpG Islands

Specimens and Objects: Endoscopic gastric biopsies with low-grade dysplasia at baseline either progressed to gastric carcinoma or persisted in dysplasia at the corresponding sites during the 5-year follow-up were selected for detection of p16 promoter methylation. All of biopsy samples (n=21) of dysplasia that progressed to gastric carcinoma was used if sections were available from paraffin blocks in which the tissue had been embedded. An equal number of samples (n=21) of dysplasia that persisted as dysplasia was selected from a tissue block archive, according to the pathological grade, sampling site, age, and sex of the patient. Distilled water and genomic DNA of human gastric carcinoma (p16 unmethylated, by bisulfite-sequencing) or genomic DNA of lymphocytes were used as templates for negative controls. Genomic DNA of p16-methylated human gastric carcinoma (p16 methylated, by bisulfite-sequencing) or of the colon cancer cell line RKO was used as a positive control.

Harvesting Cells: All sections from the margin of the gastric tissue biopsies embedded in paraffin were collected into 1.5 ml microcentrifuge tubes for each sample. The collected tissue sections were dewaxed by xylene, and rehydrated with graded ethanol before extraction of DNA samples.

Extraction of Genomic DNA: The collected sections were mixed with 300 μl of lysis buffer (10 mM Tris.Cl/pH7.6, 10 mM NaCl, 10 mM EDTA, and 0.5% SDS) and 10 μl of proteinase K solution (20 mg/ml), digested at 37° C. or 55° C. overnight. Then, the digestion was incubated at 100° C. to inactivate proteinase K. About 10 ng to 50 ng genomic DNA was extracted from each sample.

Chemical Modification of Unmethylated Cytosines:

Add 50 μl of distilled sterile water into the 1.5 ml microcentrifuge tube with the above extracted genomic DNA.

To denature double stranded DNA, add 5.5 μl of 3M NaOH, mix, incubate at 50° C.˜55° C. for 15 min.

Add 30 μl of fresh 10 mM hydroquinone, mix.

To sulfite treat the DNA, add 520 μl of fresh 1.5 M Na₂S₂O₅ (equal to 3M NaHSO₃), mix. Cover the top of reaction with 200 μl of mineral oil to prevent evaporation of reaction. Incubate at 50° C. for 16 h to deaminate unmethylated cytosines.

Remove mineral oil. Purify the modified DNA with Wizard DNA Clean-Up System (Promega A7280) as per the kit instructions: Attach syringe to minicolumn and insert the tip of column into the vacuum manifold, one set for one sample. Add 1 ml of resin solution suspended at 30° C. into microcentrifuge tube, pipette resin/DNA mix down and up, transfer the mix into column-syringe assembly, remain 5 min. Apply a vacuum to draw the solution through the column. DNA-resin binding complex was retained on the column. Break the vacuum. To wash the complexes in the column, add 2 ml of 80% isopropanol to the syringe, and reapply a vacuum to draw the solution through the column. Dry the DNA-resin complexes by continuing to draw a vacuum for 30 sec after the solution has been pulled through the column, transfer the column to a 1.5 ml microcentrifuge tube. Centrifuge the column at maximum speed (10,000 g) for 20 sec to remove any residual isopropanol. Transfer the column to a new microcentrifuge tube. Apply 50 μl of pre-warmed (80° C.) water and remain 5 min at 80° C. Centrifuge the column for 20 sec at maximum speed (10,000 g) to elute the bound DNA. Reapply 50 μl of pre-warmed (80° C.) water and remain 10 min at room temperature (RT). Centrifuge again. Remove and discard the column.

To complete the modification, add 11 μl of 3M NaOH, mix, remain 5 min at RT.

To precipitate DNA and remove NaOH, add 166 μl of SM NaOAC and 750 μl of 100% cold ethanol, mix, store at −20° C. for 2 h. Centrifuge at 10,000 g for 30 min. Discard solution. Add 200 μl of 80% cold ethanol to wash DNA. Centrifuge again. Discard solution.

Resuspend the DNA in 3 to 6 μl of sterile water or TE buffer. Use immediately or store at −20° C.

Design PCR primers: According to the modified sequences (SEQ ID NO: 1, SEQ ID NO: 2, SEQ ID NO: 3, and SEQ ID NO: 4) of methylated and unmethylated p16 CpG islands, design methylated-sequence-specific primers (sense, 5′-ttattagagg gtgggcgga tCgC-3′; antisense, 5′-GaccccGaac cGcGaccGta a-3′) and unmethylated-sequence-specific primers (sense, 5′-ttattagagg gtggggTgga tTgT-3′; antisense, 5′-cAaccccAaa ccAcAaccAt aa-3′), respectively. These regions of the artificial sequences containing frequent uracils (U) and CpG or UpG are selected for the complementary sequence of the 3′ end of each primer.

PCR Amplification: the modified templates of methylated p16 CpG islands were amplified by hot-start PCR. PCR products of the target sequence could be displayed by any chromatography technologies such as agarose gel, PAGE gel, HPLC, etc. PCR products of the methylated templates were also confirmed by sequencing. If PCR products of the methylated templates cannot be amplified from the testing samples, PCR products of the unmethylated templates should be amplified further to exclude the failures of DNA extraction, modification, and purification. Both positive control and negative control samples are used to exclude possible contamination or failures of amplification.

Results: Aberrant p16 methylation was observed in 5 of 21 samples of dysplasia that progressed to gastric carcinoma, but in none of 21 samples without progression (p=0.048, 2-sides). Sequencing results confirmed that all CpG sites were methylated in the analyzed sequence from these five p16-methylated cases. Unmethylated p16 CpG islands were detected in all of the samples without p16 methylation.

Conclusions: The present assay can specifically predict the malignant potential of gastric dysplasia. Aberrant p16 methylation was not observed in any samples of dysplasia that did not progress (Specificity, 100%). The sensitivity for detection of malignant potential of all samples of dysplasia that progressed to gastric carcinomas is only 24%. However, the sensitivity for detection of malignant potential of these samples of p16-methylated dysplasia is very high, because all 5 patients with p16-methylated gastric dysplasia progressed to gastric carcinomas at the sampling sites of their stomachs within the following five years (Sensitivity, 100%).

Alternative Protocols: aberrant p16 methylation status is also detectable in the genomic DNA of cells collected from fasting gastric juice.

All the persons working on these fields understand that the malignant potential of dysplasia can be detected by any methods that can be used for detection of methylation of p16 CpG islands. The example presented above is intended to illustrate but not limit the invention.

What is claimed is:

1. A method for in vitro detection of malignant potential of dysplasia, comprising the steps of: (a) extraction of genomic DNA from cells in a sample of tissue or body liquid; (b) detection of methylation state of p16 CpG islands in genomic DNA, by amplification with the artificial DNA sequences of SEQ ID NOs: 1-4; and (c) evaluation of malignant potential of the tested tissue or body liquid based upon presence of artificial sequences corresponding to methylated and unmethylated p16 CpG islands after chemical modification.

2. A method for in vitro detection of malignant potential of dysplasia of claim 1 wherein the methylation state of p16 CpG islands is analyzed by methylation-specific PCR (MSP).

3. A method for in vitro detection of malignant potential of dysplasia of claim 2 wherein methylated-sequence specific primers are complementary to any part of the artificial sequence SEQ ID NO: 1 or SEQ ID NO: 3, or wherein unmethylated-sequence specific primers are complementary to any part of the artificial sequence SEQ ID NO: 2 or SEQ ID NO: 4.

4. An artificial nucleotide having a sequence corresponding to the antisense sequence of methylated p16 CpG islands of SEQ ID NO: 1.

5. An artificial nucleotide having a sequence corresponding to the antisense sequence of unmethylated p16 CpG islands of SEQ ID NO: 2.

6. An artificial nucleotide having a sequence corresponding to the sense sequence of methylated p16 CpG islands of SEQ ID NO: 3.

7. An artificial nucleotide having a sequence corresponding to the sense sequence of unmethylated p16 CpG islands of SEQ ID NO: 4.


SEQUENCE LISTING (8 sequences)

SEQ ID NO: 1 - DNA, 359 bp, Artificial Sequence
  Description of Combined DNA/RNA Molecule Synthetic nucleotide sequence
agaggaggggutggutggtuauuagagggtggggcggaucgcgtgcgutcggcggutgcggagagggggagaguagguagcgggcggcggggaguaguatggagucggcggcggggaguaguatggaguuttcggutgautggutgguuacggucgcgguucggggtcgggtagaggaggtgcgggcgutgutggaggcgggggcgutguuuaacguaucgaatagttacggtcggaggucgatuuaggtgggtagagggtutguagcgggaguaggggatggcgggcgaututggaggacgaagtttguaggggaattggaatuaggtagcguttcgattutucggaaaaaggggagg

SEQ ID NO: 2 - DNA, 359 bp, Artificial Sequence
  Description of Combined DNA/RNA Molecule Synthetic nucleotide sequence
agaggaggggutggutggtuauuagagggtgggguggauugugtgugutugguggutguggagagggggagaguagguagugggugguggggaguaguatggaguuggugguggggaguaguatggaguuttuggutgautggutgguuaugguugugguuuggggtugggtagaggaggtgugggugutgutggaggugggggugutguuuaauguauugaatagttauggtuggagguugatuuaggtgggtagagggtutguagugggaguaggggatggugggugaututggaggaugaagtttguaggggaattggaatuaggtaguguttugattutuuggaaaaaggggagg

SEQ ID NO: 3 - DNA, 359 bp, Artificial Sequence
  Description of Combined DNA/RNA Molecule Synthetic nucleotide sequence
uutuuuutttttucggagaatcgaagcgutauutgattuuaattuuuutguaaauttcgtuutuuagagtcguucguuatuuuutgutuucgutguagauuututauuuauutggatcgguutucgaucgtaautattcggtgcgttggguagcguuuucguutuuaguagcguucguauutuututauucgauuucgggucgcggucgtgguuaguuagtuagucgaaggutuuatgutgutuuucgucgucggutuuatgutgutuuucgucguucgutguutgututuuuuututucguagucgucgagcguacgcggtucguuuuauuututggtgauuaguuaguuuutuutut

SEQ ID NO: 4 - DNA, 359 bp, Artificial Sequence
  Description of Combined DNA/RNA Molecule Synthetic nucleotide sequence
uutuuuutttttuuggagaatugaagugutauutgattuuaattuuuutguaaauttugtuutuuagagtuguuuguuatuuuutgutuuugutguagauuututauuuauutggatugguutuugauugtaautattuggtgugttggguaguguuuuuguutuuaguaguguuuguauutuututauuugauuucggguugugguugtgguuaguuagtuaguugaaggutuuatgutgutuuuuguuguuggutuuatgutgutuuuuguuguuugutguutgututuuuuututuuguaguuguugaguguauguggtuuguuuuauuututggtgauuaguuaguuuutuutut

SEQ ID NO: 5 - DNA, 23 bp, Artificial Sequence
  Description of Artificial Sequence Synthetic primer
ttattagagggtgggcggatcgc

SEQ ID NO: 6 - DNA, 21 bp, Artificial Sequence
  Description of Artificial Sequence Synthetic primer
gaccccgaaccgcgaccgtaa

SEQ ID NO: 7 - DNA, 24 bp, Artificial Sequence
  Description of Artificial Sequence Synthetic primer
ttattagagggtggggtggattgt

SEQ ID NO: 8 - DNA, 22 bp, Artificial Sequence
  Description of Artificial Sequence Synthetic primer
caaccccaaaccacaaccataa